(19) United States
(12) Patent Application Publication
(10) Pub. No.: US 2005/0277615 A1
(43) Pub. Date: Dec. 15, 2005

(54) Pharmaceutical compositions having anti-inflammatory activity

(75) Inventors: Pnina Fishman, Herzliya (IL); Sara Bar Yehuda, Rishon Le Zion (IL); Lea Madi, Rishon Le Zion (IL)

Correspondence Address: BROWDY AND NEIMARK, P.L.L.C.;624 NINTH STREET, NW, SUITE 300, WASHINGTON, DC, 20001-5303, US

(73) Assignee: CAN-FITE BIOPHARMA LTD., Petach Tikva (IL)

(21) Appl. No.: 10/846,918

(22) Filed: May 17, 2004

(51) Int. Cl. A61K 31/7076

(52) U.S. Cl. 514/46

(57) ABSTRACT

An anti-inflammatory pharmaceutical composition comprising as active ingredient a compound of general formula (I): 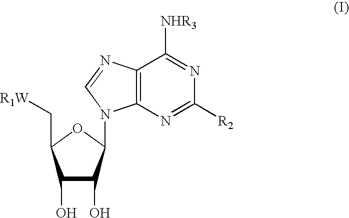 wherein W represents oxygen or sulfur atoms;
R₁ represents lower alkyl or lower cycloalkyl;
R₂ represents halogen, alkenyl, alkynyl or alkylidenhydrazino;
R₃ represents a lower alkyl, lower cycloalkyl, aryl, (ar)alkyl or anilide, said cycloalkyl, aryl and (ar)alkyl may be substituted with one or more of the groups selected from halogen, hydroxyl, hydroxyalkyl; and a pharmaceutically acceptable additive. The composition may be used to threat diseases such as multiple sclerosis, rheumatoid arthritis and Crohn's disease.

### FIELD OF THE INVENTION

This invention relates to pharmaceutical compositions having anti-inflammatory activity.

### BACKGROUND OF THE INVENTION

A list of prior art which is considered to be pertinent for describing the state of the art in the field of the invention appears at the end of the description before the claims. Acknowledgement of these references herein will be made by indicating their number from the list of publications.

Adenosine acts extracellularly via activation of specific membrane-bound receptors called P₁-purinoceptors. These adenosine receptors can be divided into four subclasses, A₁, A_{2A}, A_{2B} and A₃ receptors. All four classes are coupled to the enzyme adenylate cyclase. Activation of the adenosine A₁ and A₃ receptors leads to an inhibition of adenylate cyclase, while activated A_{2A} and A_{2B} receptors stimulate adenylate cyclase. The adenosine receptors are ubiquitously distributed throughout the body. As a consequence, ligands need to be highly selective in their action with respect to receptor subtype and tissue to be of therapeutic value.

Receptor subtype selectivity can be achieved by substituting the adenosine molecule. For example modification at the N⁶ position of adenosine is well tolerated. N⁶-substituents such as cyclopentyl enhance adenosine A₁ receptor selectivity relative to the other subtypes,^{1,2} while a 3-iodobenzyl group induces adenosine A₃ receptor selectivity.³⁻⁵ Bulky substituents such as (ar)alkylamino,⁶ alkylidenehydrazino⁷ and alkynyl,⁸ at the 2-position of the adenine moiety yield selectivity for the adenosine A_{2A} receptor compared to A₁. Only more recently, the 2-(ar)alkynyl adenosine derivatives have been evaluated at the adenosine A₃ receptor. Quite surprisingly, some of these compounds appeared to be selective for the adenosine A₃ receptor rather than for A_{2A}.^{9,10}

Tissue selectivity is often the result of partial agonism, which may reduce the extent of side effects.^{11,12} Due to differences in receptor-effector coupling in various tissues selectivity of action in vivo may be achieved. Partial agonists for the adenosine receptors may be of use as antipsychotic drugs, e.g., via stimulation of the adenosine A_{2A} receptor that leads to inhibition of dopamine D₂ receptors in the basal ganglia,^{13,14} and as cardio- and cerebroprotective agents via the adenosine A₃ receptor when chronically administered.^{15,16}.

Multiple sclerosis (MS) is a chronic, progressive, degenerative disease of the central nervous system (CNS), and particularly of the “white matter” tissue. It is considered an autoimmune disease characterized by inflammation and demyelination of the CNS leading to chronic neuralgic disturbances. Autoantibodies are generated by the immune system against antigens of myelin proteins such as myelin basic protein (MBP) which envelops the spinal cord.

Experimental autoimmune encephalomyelitis (EAE) is the commonly used animal model for MS. It may be induced in wild-type animals such as rodents by inoculation, or appear spontaneously in genetically susceptible strains.

U.S. Pat. No. 5,506,214 (Beutler) discloses treatment of patients having MS with therapeutic agents containing substituted adenine derivatives such as 2-chloro-2′-deoxyadenosine (CdA). Treatment with CdA was shown to markedly ameliorate the disease condition. CdA was found to be a putative partial agonist at A1 receptors, as described in Siddiqi, S. M. et al, (1995) J. Med. Chem. 38:1174-1188. The Kᵢ values of CdA for the various adenosine receptors were 7.4 μM at the A₁ receptor, 20 μM at the A2a receptor and 207 μM at the A3 receptor.

U.S. Patent Application No. 20020094974 (Castelhano, et al) discloses new N-6 substituted 7-deazapurine derivatives which are A3 adenosine receptor antagonists. These compounds may be used for treating diseases associated with the A3 adenosine receptor, including neurological disorders such as MS.

Rheumatoid arthritis is a common rheumatic disease, affecting more than two million people in the United States alone. The disease is three times more prevalent in women as in men but afflicts all races equally. The disease can begin at any age, but most often starts between the ages of forty and sixty. In some families, multiple members can be affected, suggesting a genetic basis for the disorder. The cause of rheumatoid arthritis is unknown. Even though infectious agents such as viruses, bacteria, and fungi have long been suspected, none has been proven as the cause. It is suspected that certain infections or factors in the environment might trigger the immune system to attack the body's own tissues, resulting in inflammation in various organs of the body. Regardless of the exact trigger, the result is an immune system that is geared up to promote inflammation in the joints and occasionally other tissues of the body. Lymphocytes are activated and cytokines, such as tumor necrosis factor/TNF and interleukin-1/IL-1 are expressed in the inflamed areas.

The clinical expression of rheumatoid arthritis is manifested by chronic inflammation of the joints, the tissue surrounding the joints such as the tendons, ligaments, and muscles, as well as other organs in the body such as the eyes. The inflammation process of causes swelling, pain, stiffness, and redness in the joints. In some patients with rheumatoid arthritis, chronic inflammation leads to the destruction of the cartilage, bone and ligaments causing deformity of the joints.

### SUMMARY OF THE INVENTION

The present invention provides pharmaceutical compositions for the treatment of inflammatory diseases comprising as active ingredient an effective amount of one or more of a compound of the general formula (I):

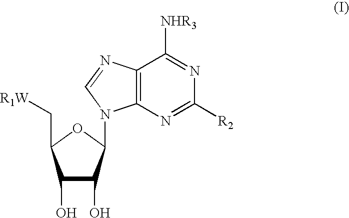

in which
W represents an oxygen, or sulfur atom;
R₁ represents a lower alkyl or lower cycloalkyl;
R₂ represents a halogen, loweralkenyl, lower alkynyl or lower alkylidenehydrazino;
R₃ represents a lower alkyl, lower cycloalkyl, (ar)alkyl, aryl or anilide, said cycloalkyl, aryl or (ar)alkyl may be substituted with one or more halogen atom(s), hydroxy, hydroxyalkyl;
or a salt of said compound.

The compounds which may be used in the pharmaceutical compositions of the invention are disclosed in WO 02/070532, whose entire contents are incorporated by reference.

By the term “alkyl” which may be used herein interchangeably with the term “lower allyl”. it is meant any saturated carbohydrate, either linear or branched chain comprising from 1 to about 10 carbon atoms in the backbone.

Accordingly, the terms “alkenyl” and “alkynyl” which are also used interchangeably and respectively with the terms “lower alkenyl” and “lower alkynyl” refer to linear or branched carbohydrates comprising from 2 to 10 carbon atoms in the backbone, wherein at least two of the carbon atoms are connected via a double or triple bond, respectively.

Thus, it is to be understood that the term “lower” when used a prefix for defining a carbohydrate, refers to any carbohydrate having in its backbone no more than 10 carbon atoms.

When referring to salts of the compound of the present invention it is meant any physiologically acceptable salt. The term “physiologically acceptable salt” refers to any non-toxic alkali metal, alkaline earth metal, and ammonium salts commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the compounds of this invention with a suitable organic or inorganic acid. The acid addition salts are those which retain the biological effectiveness and properties of the free bases and which are not biologically or otherwise undesirable. Examples include acids are those derived from mineral acids, and include, inter aila, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g. p-toluenesulphonic, acids.

According to one preferred embodiment, W is a sulfur atom, R₁ is a lower alkyl selected from the group consisting of methyl, ethyl, n- and i-propyl; R₂ is an alkynyl group; and R₃ is a hydrogen. According to this embodiment, R₂ is preferably 1-hexynyl.

Specific compounds used in the present invention include:
5′-Deoxy-2-iodo-5methylthioadenosine; (compound 33 hereinafter);
5′-Deoxy-2-iodo-5′-ethylthioadenosine (compound 34 hereinafter);
5′-Deoxy-2-iodo-5′-propylthioadenosine (compound 35 hereinafter).
5′-Deoxy-2-iodo-5′-isopropylthioadenosine (compound 36 hereinafter);
5′-Deoxy-2-(1-hexynyl)-5′-methylthioadenosine (compound 37 hereinafter);
5′-Deoxy-2-(1-hexynyl)-5′-ethylthioadenosine (compound 38 hereinafter);
5′-Deoxy-2-(1-hexynyl)-5′-propylthioadenosine (compound 39 hereinafter); and
5′-Deoxy-2-(1-hexynyl)-5′-isopropylthioadenosine (compound 40 hereinafter).

According to a particularly preferred embodiment, the active ingredient comprises compound 37.

A further aspect of the invention relates to use of a compound of general formula (I) for the preparation of a pharmaceutical composition for administration to a subject suffering from an inflammatory disease.

A still further aspect of the invention relates to a method for treating an inflammatory disease in a subject suffering therefrom comprising administrating to said subject a pharmaceutical composition comprising as active ingredient a compound of general formula (I).

Inflammatory diseases which may be treated using the composition of the invention are well known by the skilled man of the art, and include, but are not limited to, multiple sclerosis (MS), rheumatoid arthritis and Crohn's disease.

The “effective amount” for purposes herein is determined by such considerations as may be known in the art. The amount must be effective to achieve the desired anti-inflammatory effect. For example, with respect to MS, the present invention refers to any improvement in the clinical symptoms of the disease, and/or a reduction in the rate of deterioration or the relapse rate of the MS patient, as well as any improvement in the well being of the patients. For example, an improvement may be manifested by one or more of the following: decrease in muscle weakness, decrease in muscle spasms, reduction of spasticity, improvement of balance and improvement in memory.

The effective amount depends, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

The terms “treat”, “treating” and “treatment” refer to the administering of a therapeutic amount of the compound or composition of the present invention which is effective to ameliorate undesired symptoms associated with a disease, to prevent the manifestation of such symptoms before they occur, to slow down the progression of a disease, to slow down the deterioration of symptoms, to slow down the irreversible damage caused by the chronic stage of a disease, to lessen the severity or cure a disease, to improve survival rate or more rapid recovery, to prevent the disease from occurring, or a combination of two or more of the above.

The pharmaceutical composition of the present invention may further comprise pharmaceutically acceptable additives.

Further, the term “pharmaceutically acceptable additives” used herein refers to any substance combined with said compound and include, without being limited thereto, diluents, excipients, carriers, solid or liquid fillers or encapsulating materials which are typically added to formulations to give them a form or consistency when it is given in a specific form, e.g. in pill form, as a simple syrup, aromatic powder, and other various elixirs. The additives may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc.

Preferably, the additives are inert, non-toxic materials, which do not react with the active ingredient of the invention. Yet, the additives may be designed to enhance the binding of the active agent to its receptor. Further, the term additive may also include adjuvants, being substances affecting the action of the active ingredient in a predictable way.

The additives can be any of those conventionally used and are limited only by chemico-physical considerations, such as solubility and lack of reactivity with the compound of the invention, and by the route of administration.

The active agent of the invention may be administered orally to the patient. Conventional methods such as administering the compound/s in tablets, suspensions, solutions, emulsions, capsules, powders, syrups and the like are usable.

For oral administration, the composition of the invention may contain additives for facilitating oral delivery of the compound/s of the invention. Formulations suitable for oral administration can consist of (a) liquid solutions, such as an effective amount of the compound dissolved in diluents, such as water, saline, syrup, juice, etc.; (b) capsules, sachets, tablets, lozenges, and troches, each containing a predetermined amount of the active ingredient, as solids or granules; (c) soft gel capsules encapsulating a solution or a suspension of the active ingredient; (d) powders; (e) suspensions in an appropriate liquid; and (f) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodiumk talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active agent in a flavor, usually sucrose and acacia or tragacanth, as well as pastilles comprising the active ingredient in an inert base, such as gelatin and glycerin, or sucrose and acacia, emulsions, gels, and the like. Such additives are known in the art.

Alternatively, the compound/s may be administered to the patient parenterally. In this case, the composition will generally be formulated in a unit dosage injectable form (solution, suspension, emulsion). Pharmaceutical formulation suitable for injection may include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, lipid polyethylene glycol and the like), suitable mixtures thereof; a vegetable oil such as cottonseed oil, sesame oil, olive oil, soybean oil, corn oil, sunflower oil, or peanut oil; a fatty acid esters such as ethyl oleate and isopropyl myristate and variety of other solvent systems as known per se. The carrier may be chosen based on the physical and chemical properties of the active agent.

In case the active ingredient has poor water solubility, and an oily carrier is therefore used, proper fluidity can be maintained, for example, by the use of a emulsifiers such as phospholipids, e.g. lecithin or one of a variety of other pharmaceutically acceptable emulsifiers. As known per se, the proper choice if a surfactant and the treatment conditions may also permit to control the particle size of the emulsion droplets.

Suitable soaps for use in parenteral formulations, in case the active ingredient has poor water solubility, include fatty alkali metal, ammonium, and triethanolamine salts, and suitable detergents include oleic acid, stearic acid, and isostearic acid. Ethyl oleate and isopropyl myristate are examples of suitable fatty acid esters.

Suitable detergents for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

Further, in order to minimize or eliminate irritation at the site of injection, the compositions may contain one or more nonionic surfactants having a hydrophile-lipophile balance (HLB) of from about 12 to about 17. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The choice of an additive will be determined in part by the particular compound of the present invention, as well as by the particular method used to administer the composition.

Notwithstanding the above, the composition of the present invention may include one or more of the compounds of the present invention and may be comprise other biologically active substances, to provide a combined therapeutic effect.

The compounds and compositions of the present invention as set forth hereinabove and below are administered and dosed in accordance with good medical practice, taking into account the clinical conditions of the individual patient, the site and method of administration, scheduling of administration, individual's age, sex, body weight and other factors known to medical practitioners.

The dose may be single doses or multiple doses over a period of several days. The treatment generally has a length proportional to the length of the disease process and drug effectiveness and the individual species being treated. Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments, until the optimum effect under the circumstances is reached. Exemplary daily dosages range from about 1 μg/kg body weight to about 10,000 μg/kg body weight of the subject being treated. A preferred dosage range may be between about 1 μg/kg, typically between about 4 μg/kg and occasionally between about 8 μg/kg body weight, to about 1,000 μg/kg, typically to about 400 and occasionally to about 100 μg/kg body weight.

The invention has been described in an illustrative manner, and it is to be understood that the terminology which has been used, is intended to be in the nature of words of description rather than of limitation. Obviously, many modifications and variations of the present invention are possible in light of the above teaching. It is therefore, to be understood that within the scope of the appended claims, the invention may be practiced otherwise than as specifically described hereinafter.

Throughout the description various publications are referred to by a number. Full citations of the publications are listed at the end of the description before the claims.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a bar graph illustrating the clinical EAE symptoms of rats injected with CF402 as compared to control rats;

FIG. 2 illustrates a Western blot of a protein extract from spinal cord of the CF402 treated and control rats of FIG. 1;

FIG. 3 is a line plot of a second experiment illustrating the clinical EAE symptoms of rats injected with CF402 as compared to control rats; and

FIG. 4 is a bar graph illustrating % of weight loss of rats induced with colitis injected with CF402 as compared to control rats as a function of time.

### DETAILED DESCRIPTION OF THE INVENTION

Materials and Methods

5′-deoxy-2-(1-hexynyl)-5′-methylthioadenosine (referred to also as CF402) was synthesized as described in WO 02/070532 and in van Tilburg, E. W., et al, J. Med. Chem. (2002) 45:420-429 (with reference to compound 37).
Chemical Structure:

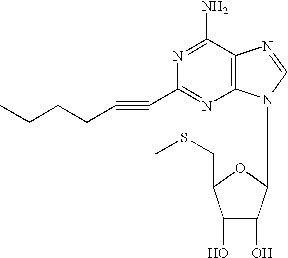

Molecular Formula: C₁₇H₂₃IN₅O₃S

Molecular Weight: 377.46

Description

The standard material is a yellowish-light brown powder. The material was visually inspected against a white background.

Melting Point

Using a Büchi capillary melting point apparatus, the melting point of CF402 was determined to be in the range of 64-67° C.

Solubility Profile

A preliminary, qualitative solubility study of CF402 was completed. The solubility of CF402 in water and DMSO at 1 mg/ml and at ambient temperature is shown in Table 1 below.

TABLE 1
Solubility Profile

| Solvent | Result |
|---|---|
| Water | Insoluble |
| DMSO | Soluble |

Synthetic Protocol

General. To a solution of the appropriate 5′-alkylthio-5′-deoxy-2-iodoadenosine (0.92 mmol) in 7 mL dry acetonitrile and 7 mL triethylamine under a nitrogen atmosphere was added CuI (0.07 mmol, 13.3 mg), PdCl₂ (0.05 mmol, 8.47 mg) and Ph₃P (0.11 mmol). To the suspension was added 1 -hexyn (4.45 mmol, 511 μL) and the mixture was stirred overnight under nitrogen atmosphere. The light brown solution was filtered and concentrated. The residu was extracted with water and EtOAc (3×50 mL), the organic layer was dried, concentrated and purified by column chromatography.

5′-Deoxy-2-(1-hexynyl)-5′-methylthioadenosine (CF402). The reaction was carried out with 5′-deoxy-2-iodo-5′-methylthioadenosine (480 mg, 1.13 mmol). The mixture was purified by column chromatography (eluent CH₂Cl₂ to 10% MeOH in CH₂Cl₂).Yield 257 mg (0.68 mmol, 60%); mp 64-67° C; R_{f} 0.28 (10% MeOH in CH₂Cl₂). An aliquot of the product was recrystallised from methanol for analytical purposes; ¹H NMR (DMSO-d₆) δ 8.37 (s, 1H, H-8), 7.39 (s, 2H, NH₂), 5.85 (d, J=6.18 Hz, 1H, H-1′), 5.49 (d, J=6.18 Hz, 1H, OH-2′), 5.32 (d, J=4.81 Hz, 1H, OH-3′), 4.67 (q, J=5.49 Hz, 1H, H-2′), 4.12-3.95 (m, 1H, H-3′), 4.12-3.95 (m, 1H, H-4′), 2.84 (t, J=5.49 Hz, 2H, H-5′), 2.40 (t, J=6.68 Hz, 2H, ≡CCH₂), 2.05 (s, 3H, SCH₃), 1.55-1.32 (m, 4H, ≡CCH₂CH₂CH₂), 0.90 (t, J=6.18 Hz, 3H, CH₃).

General. The appropriate 6-chloro-2-iodo-9-(2,3-di-O-acetyl-5-alkylthio-5-deoxy-β-D-ribofuranosyl)-purine (5.33 mmol) was stirred with 50 mL EtOH/NH₃ for 64 h. The mixture was concentrated and purified by column chromatography.

5′-Deoxy-2-iodo-5′-methylthioadenosine. The reaction was carried out with 6-chloro-2-iodo-9-(2,3-di-O-acetyl-5-deoxy-5-methylthio-β-D-ribofuranosyl)-purine (3.99 g, 7.58 mmol). The mixture was purified by column chromatography (10% MeOH in CH₂Cl₂). Yield 2.21 g (5.22 mmol, 69%), mp 90-93° C.; R_{f} 0.24 (10% MeOH in CH₂Cl₂). The product was recrystallised from EtOAc; ¹H NMR (DMSO-d₆) δ 8.29 (s, 1H, H-8), 7.71 (bs, 2H, NH₂), 5.79 (d, J=5.84 Hz, 1H, H-1′), 5.52 (d, J=6.52 Hz, 1H, OH-2′), 5.35 (d, J=5.80 Hz, 1H, OH-3′), 4.69 (m, 1H, H-2′), 4.11-4.02 (m, 1H, H-3′), 4.11-4.02 (m, 1H, H-4′), 2.85-2.80 (m, 2H, H-5′), 2.06 (s, 3H, SCH₃); MS m/z 424 (M+H)⁺; Anal. (C₁₁H₁₄IN₅O₃S.0.35 EtOAc) C, H, N.

General diazotization method. Isopentylnitrite (23.2 mmol, 3.10 mL) was added to a mixture of the appropriate 2-amino-6-chloro-9-(2,3-di-O-acetyl-5-alkylthio-5-deoxy-β-D-ribofuranosyl)-purine (7.49 mmol), I₂ (7.49 mmol, 1.90 g), CH₂I₂ (77.5 mmol, 6.24 mL) and CuI (7.87 mmol, 1.50 g) in 40 mL tetrahydrofuran. The dark brown solution was refluxed (under intensive cooling) for 40-60 minutes and then cooled to room temperature. The mixture was filtered and the filtrate was concentrated in vacuo. The residue was dissolved in CH₂Cl₂ and extracted with a saturated Na₂S₂O₃ solution, until the colour disappeared. The organic layer was dried and concentrated. The brownish oil was purified by column chromatography.

### 6-Chloro-2-iodo-9-(2,3-di-O-acetyl-5-deoxy-5-methylthio-β-D-ribofuranosyl)-purine

The reaction was carried out with 2-amino-6-chloro-9-(2,3-di-O-acetyl-5-deoxy-5-methylthio-β-D-ribofuranosyl)-purine (3.83 g, 9.21 mmol). The mixture was purified by column chromatography (eluens CH₂Cl₂-5% MeOH in CH₂Cl₂). Yield 3.99 g (7.58 mmol, 82%), R_{f} 0.62 (5% MeOH in CH₂Cl₂); ¹H NMR (DMSO-d₆) δ 8.84 (s, 1H, H-8), 6.27 (d, J=5.49 Hz, 1H, H-1′), 5.96 (t, J=5.49 Hz, 1H, H-2′), 5.58 (t, J=5.49 Hz, 1H, H-3′), 4.37-4.32 (m, 1H, H-4′), 2.98 (d, J=6.86 Hz, 2H, H-5′), 2.12, 2.07 (2×s, 6H, 2×COCH₃), 2.02 (s, 3H, SCH₃).

General chlorination procedure. To a suspension of the appropriate 2′,3′-di-O-acetyl-5′-alkylthio-5′-deoxyguanosine (19.3 mmol, predried) and tetraethylammonium chloride (6.48 g, 39.1 mmol; predried in vacuo at 80° C.) in acetonitrile (40 mL) were added N,N-dimethylaniline (2.52 mL, 20.0 mmol, dried and distilled from KOH), and phosphoryl chloride (POCl₃, 10.95 mL, 0.12 mol, freshly distilled) at room temperature. The flask was placed in an oil bath preheated at 100° C. and the solution was refluxed for 10-15 minutes. Volatile materials were evaporated immediately in vacuo. The resulting yellow foam was dissolved in CH₂Cl₂ (100 mL) and stirred vigorously for 15 minutes with crushed ice. The layers were separated and the aqueous phase was extracted with CH₂Cl₂ again (75 mL). The combined organic layers were kept cold by addition of crushed ice and washed with cold water (3×75 mL), 5% NaHCO₃/H₂O to pH 7, dried over MgSO₄ and filtered. The residue was purified by column chromatography.

2-Amino-6-chloro-9-(2,3-di-O-acetyl-5-deoxy-5-methylthio-β-D-ribofuranosyl)-purine. The reaction was carried out with 2′,3′-di-O-acetyl-5′-deoxy-5′-methylthioguanosine (5.96 g, 15.0 mmol). The mixture was purified by column chromatography (eluens EtOAc:PE40/60=1:1 to 2:1). Yield 3.83 g (9.21 mmol, 62%), R_{f} 0.28 (EtOAc:PE40/60=2: 1). ¹H NMR (DMSO-d₆) δ 8.40 (s, 1H, H-8), 7.08 (bs, 2H, NH₂), 6.10-5.99 (m, 2H, H-1′, H-2′), 5.49-5.45 (m, 1H, H-3′), 4.31-4.24 (n, 1H, H-4′), 2.96 (pd, J=6.86 Hz, 2H, H-5′), 2.12, 2.06 (2xs, 6H, COCH₃), 1.97 (s, 3H, SCH₃).

General acetylation procedure. To a suspension of the appropriate 5′-alkylthio guanosine derivative (0.46 mmol) and 4-dimethylaminopyridine (DMAP; 0.03 mmol) in a mixture of acetonitrile (5.7 mL) and triethylamine (154 μl, 1.1 mmol) was added acetic anhydride (95 μL, 1 mmol) at room temperature. The mixture was stirred for 1 h until the solution became clear. Methanol (10 mL) was added and the solution was stirred for 5-10 minutes, concentrated in vacuo and stirred with isopropanol. The white slurrie obtained was filtered and subsequently stirred with hexane. The white precipitate was filtered and dried.

2′,3′-di-O-Acetyl-5′-deoxy-5′-methylthioguanosine. The reaction was carried out with 5′-deoxy-5′-methylthioguanosine (10.4 g, 33.2 mmol). Yield 10.5 g (26.4 mmol, 79%), ¹H NMR (DMSO-d₆) δ 7.98 (s, 1H, H-8), 6.59 (bs, 2H, NH₂), 5.99-5.90 (m, 1H, H-1′), 5.99-5.90 (m, 1H, H-2′), 5.43 (t, J=3.78 Hz, 1H, H-3′), 4.24 (pq, J=3.19 Hz, 1H, H-4′), 2.96-2.88 (m, 2H, H-5′), 2.11, 2.07 (2×s, 6H, 2×COCH₃), 2.00 (s, 3H, SCH₃).

General procedure for the syntheses of 5′-alkylthio derivatives. The appropriate thiol (3.32 mmol) was dissolved in 10 mL 2 M NaOH. After stirring, 5′-chloro-5′-deoxyguanosine (100 mg, 0.33 mmol) was slowly added. The mixture was refluxed for 2-2.5 h and then cooled to room temperature. It was acidified with acetic acid and a white precipitate was formed. The precipitate was filtered and dried.

5′-Deoxy-5′-methylthioguanosine. The reaction was carried out with sodium thiomethoxide (27.42 g, 0.39 mol) and 5′-chloro-5′-deoxyguanosine (11.8 g, 39.1 mmol). Yield 10.41 g (33.2 mmol, 85%), ¹H NMR (DMSO-d₆) δ 7.85 (s, 1H, H-8), 7.23 (bs, 2H, NH₂), 5.68 (d, J=6.18 Hz, 1H, H-1′), 4.53-4.51 (m, 1H, H-2′), 4.05-3.99 (m, 1H, H-3′), 3.99-3.95 (m, 1H, H-4′), 2.78 (t, J=6.52 Hz, 2H, H-5′), 1.67 (s, 3H, CH₃).

5′-Chloro-5′-deoxyguanosine. Guanosine (43.5 g, 0.15 mol) was dissolved in hexamethylphosphorictriamide (HMPA, 40 mL, 0.23 mol). Thionyl chloride (61.5 mL, 0.85 mol) was added in 1 h. The mixture was stirred at ambient temperature for 1 h, diluted with water and chromatographed on Dowex 50 W (H⁺). After washing with water (350 mL), the product was collected by eluting 5% aqueous ammonia (350 mL). The fraction was concentrated in vacuo. Yield 40 g (0.13 mol, 86%), ¹H NMR (DMSO-d₆) δ 10.53 (bs, 1H, NH), 7.89 (s, 1H, H-8), 6.50 (bs, 2H, NH₂), 5.72 (d, J=5.84 Hz, 1H, H-1′), 5.55 (d, J=6.52 Hz, 1H, OH-2′), 5.39-5.35 (m, 1H, OH-3′), 4.57 (q, J=5.15 Hz, 1H, H-2′), 4.16-4.05 (m, 1H, H-3′), 4.05-3.97 (m, 1H, H-4′), 3.86 (dq, J=11.67 Hz, 2H, H-5′).

Drug Substance Purity

An aliquot of the laboratory sample of CF402 was subjected to recrystallization and subsequently to elemental and MS analysis (Department of Analytical Chemistry, Leiden University, The Netherlands). Elemental analyses were performed for C, H, N. Results (within 0.4% of theoretical value): C₁₇H₂₃N₅O₃S.0.56 CH3OH. All high resolution mass spectra were measured on a Finnigan MAT900 mass spectrometer equipped with a direct insertion probe for EI experiments (70 eV with resolution 1000) or on a Finnigan MAT TSQ-70 spectrometer equipped with an electrospray interface for ESI experiments. Spectra were collected by constant infusion of the analyte dissolved in 80/20 methanol/H₂O. ESI is a soft ionization technique resulting in protonated, sodiated species in positive ionization mode and deprotonated species in the negative ionization mode. MS n/z 378 (M+H)⁺.

### EXAMPLE I

### Biological Evaluation of CF402

General. All compounds (CF402 and reference materials) were tested in radioligand binding assays to determine their affinities for the adenosine A₁ receptor in rat brain cortex, the A_{2A} receptor in rat striatum and the human A₃ receptor as expressed in HEK 293 cells (Table 1). For the adenosine A₁ receptor, the tritiated antagonist, [³H]-1,3-dipropyl-8-cyclopentylxanthine ([³H]DPCPX), and for the adenosine A_{2A} receptor, the tritiated antagonist [³H]ZM 241385 were used. Since radiolabeled antagonists are not commercially available for the adenosine A₃ receptor, [¹²⁵I] AB-MECA, an A₃ receptor agonist, was used. Displacement experiments were performed in the absence of GTP.

All compounds were also tested in functional assays. The ability of the compounds to either stimulate the cyclic AMP (cAMP) production through human adenosine A_{2A} receptors expressed in CHO cells or inhibit the cAMP production in human adenosine A₃ receptors expressed in HEK 293 cells was assessed.

Experimental Details

Radioligand Binding Studies. Measurements with [³H]DPCPX in the absence of GTP were performed according to a protocol published previously (Pirovano et al, Eur J Pharmacol 172 (1989) 185). Adenosine A_{2A} receptor affinities were determined according to Gao et al (Biochem Pharmacol 60 (2000) 669). Adenosine A₃ receptor affinities were determined essentially as described earlier (Van Galen et al, Mol Pharmacol 45 (1994) 1101). Briefly, assays were performed in 50/10/1 buffer (50 mM Tris/10 mM MgCl₂/1 mM ethylenediaminetetra-acetic acid (EDTA) and 0.01% 3-([3-cholamidopropyl]-dimethylammonio)-1-propanesulfonate (CHAPS)) in glass tubes and contained 50 μL of a HEK 293 cell membrane suspension (10-30 μg), 25 μL [¹²⁵I]AB MECA (final concentration 0.15 nM), and 25 μL of ligand. Incubations were carried out for 1 hr at 37° C. and were terminated by rapid filtration over Whatman GF/B filters, using a Brandell cell harvester (Brandell, Gaithersburg, Md.). Tubes were washed three times with 3 ml of buffer. Radioactivity was determined in a Beckman 5500B γ-counter. Nonspecific binding was determined in the presence of 10⁻⁵ M R-PIA.

cAMP assay A_{2A}. CHO cells expressing human adenosine A_{2A} receptors were grown overnight as a monolayer in 24 wells tissue culture plates (400 μL/well; 2×10⁵ cells/well). cAMP generation was performed in Dulbecco's Modified Eagles Medium (DMEM)/N-2-hydroxyethylpiperazin-N′-2-ethanesulfonic acid (HEPES) buffer (0.60 g HEPES/50 mL DMEM pH 7.4). To each well, washed three times with DMEM/HEPES buffer (250 μL), 100 μL DMEM/HEPES buffer, 100 μL adenosine deaminase (final concentration 5 IU/mL) and 100 μL of a mixture of rolipram and cilostamide (final concentration 50 μM each) were added. After incubation for 40 minutes at 37° C., 100 μL agonist was added. After 15 minutes at 37° C., the reaction was terminated by removing the medium and adding 200 μL 0.1 M HCl. Wells were stored at −20° C. until assay.

cAMP assay A₃. CHO cells expressing the human adenosine A₃ receptor were grown overnight as a monolayer in 24 wells tissue culture plates (400 μL/well; 2×10⁵ cells/well). cAMP generation was performed in Dulbecco's Modified Eagles Medium (DMEM)/N-2-hydroxyethylpiperazin-N′-2-ethansulfonic acid (HEPES) buffer (0.60 g HEPES/50 mL DMEM pH 7.4). To each well, washed three times with DMEM/HEPES buffer (250 μL), 100 μL adenosine deaminase (final concentration 5 IU/mL), 100 μL of a mixture of rolipram and cilostamide (final concentration 50 μM each) and 100 μL agonist (final concentration approx. 100× the Kᵢ value) were added. After incubation for 40 minutes at 37° C., 100 μL forskolin (final concentration 10 □M) was added. After 15 minutes at 37° C., the reaction was terminated by removing the medium and adding 200 μL 0.1 M HCl. Wells were stored at −20° C. until assay. The amounts of cAMP were determined after a protocol with cAMP binding protein³⁶ with the following minor modifications. As a buffer was used 150 mM K₂HPO₄/10 mM EDTA/0.2% Bovine Serum Albumine (BSA) at pH 7.5. Samples (20 μL+30 μL 0.1 M HCl) were incubated for at least 2.5 hours at 0° C. before filtration over Whatman GF/B filters. Filters were additionally rinsed with 2×2 mL TrisHCl buffer (pH 7.4, 4° C.). Filters were counted in Packard Emulsifier Safe scintillation fluid (3.5 mL) after 24 hours of extraction.

Data Analysis. Apparent Kᵢ and EC₅₀ values were computed from the displacement curves by non-linear regression of the competition curves with the software package Prism (Graph Pad, San Diego, Calif.).

Results

TABLE 1
Radioligand binding affinities of CF402 and reference adenosine
analogues at adenosine A₁, A_{2A} and A₃ receptors expressed as Kᵢ values
(±SEM in nM, n = 3) or percentage displacement
at 10 μM (CPA-A₁ agonist).
Ki (nM) or % displacement at 10⁻⁵ M

| compound | A₁^{a} | A_{2A}^{b} | A₃^{c} |
|---|---|---|---|
| CPA | 7.14 ± 2.30 | 580 ± 120 | 120 ± 15 |
| IB-MECA | 1400 ± 240 | 39% | 6.9 ± 0.2 |
| Cl-IB-MECA | 710 ± 41 | 24% | 7.2 ± 0.9 |
| CF402 | 36% | 60 ± 20 | 14.5 ± 3.4 |

^{a}Displacement of [³H]DPCPX from rat cortical membranes.
^{b}Displacement of [³H]ZM 241385 from rat striatal membranes,
^{c}Displacement of [¹²⁵I]AB MECA from the human A₃ receptor expressed in HEK 293 cells.

TABLE 2
EC₅₀ values and maximum levels of activity (Eₘₐₓ) for CF402 and
reference adenosine analogues at the A_{2A} receptor and the Eₘₐₓ values
at the A₃ receptor, as determined in cAMP assays (CGS21680-A_{2A}
agonist; NECA-adenosine agonist)..

| | Eₘₐₓ | (%) EC₅₀ (μM) | | |
|---|---|---|---|---|
| compound | A_{2A}^{a} | CHO cells A_{2A} | Eₘₐₓ (%) A₃^{b} | |
| CGS21680 | 100 | — | — | |
| NECA | 102 ± 23 | 0.04 ± 0.004 | — | |
| Cl-IB-MECA | — | — | 83 ± 2 | (10) |
| CF402 | 45 ± 6 | 0.7 ± 0.1 | 72 ± 9 | (3) |

^{a}Eₘₐₓ compared to the Eₘₐₓ of CGS21680 (±SEM, n = 3; 10 μM) in A_{2A} CHO cells;
^{b}Percentage of inhibition of forskolin-induced (10 μM) cAMP production, compared to Cl-IB-MECA. In parentheses the concentration at which concentration the effect was determined (μM, approx. 100 x Kᵢ value);
—: not determined.

### EXAMPLE II

### Induction of Experimental Autoimmune Encephalomylitis (EAE)

EAE is an inflammatory demyelinating disease of the nervous system, which serves as a model for multiple sclerosis (MS). EAE was induced by intradermal injection at the base of the tail of female Lewis rats (8 weeks old) with an emulsion consisting of the following for each rat: 100 μg myelin basic protein (MBP) from guinea pig (M2295; Sigma), 0.1 ml Complete Freund's adjuvant (CFA; F5506, Sigma), and 0.2 mg of Mycobacterium tuberculosis H37 Ra (M. tuberculosis, 3114, Difco). The emulsion was injected in two halves into the medial footpad of each hind limb of the rats. CF402 treatment (10 μg/kg, PO, BID) started at day 7 after disease induction.

The rats developed clinical EAE symptoms which were graded into the following categories: 0, no neurological symptoms; 1, loss of tail tonus and paralysis of the whole tail; 2, hind limbs weakness; 3, hind limbs paralysis; 4, quadriplegia; 5, moribund. The immunized rats developed acute monophasic EAE within 10 days after immunization.

Results

A remarkably low clinical score in the CF402 treated group in comparison to the control group was noted. The difference in the maximal clinical score between the CF402 and the control groups was significant with P<0.01 using the Student's t test (FIG. 1).

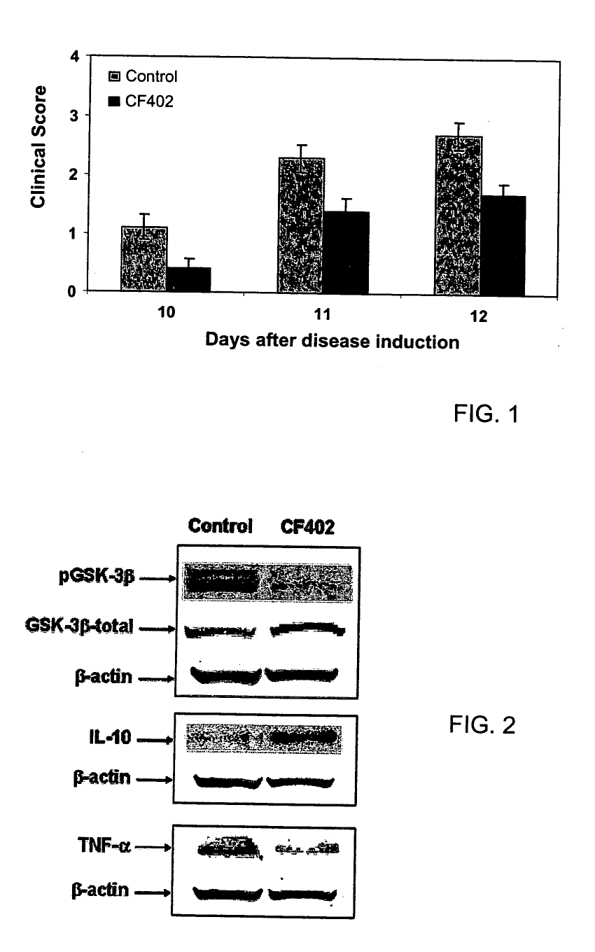

Examination of a protein extract from the spinal cord of the CF402 treated and untreated rats indicated down-regulation in the level of the pro-inflammatory cytokine TNF-α in the CF402 treated group and up-regulation in the anti-inflammatory cytokine IL-10. Also, a decrease in the phosphorylated GSK-3β protein expression level was observed in the CF402 treated group, indicating the induction of an apoptotic process in the diseased cells (FIG. 2).

### EXAMPLE III

EAE was induced by common myelin-associated proteins, MOG peptide (35-55) in female, C57B1 mice (6-8 weeks). The encephalitogenic emulsion containing MOG (300 μg/mouse) in Complete Freund's adjuvant enriched with 5 mg/mL Mycobacterium Tuberculosis was injected subcutaneously in the right flank of the mouse. A boost of the encephalitogenic emulsion was injected subcutaneously in the left flank one week later. Also, on the day of the first injection of MOG, Pertussis toxin (300 ng/mouse) was injected intraperitoneally at a volume dose of 0.1 mL/mouse. The injection of the Pertussis Toxin was repeated after 48 hours. The mice were observed daily from the 10^{th} day post-EAE induction (first injection of MOG) and the EAE clinical signs were scored as follows:. 0—No neurological signs; 1—Distal limp tail: 1.5—Complete limp tail; 2—Difficulties to return on feet when laid on the back; 3—Ataxia; 4—Early paralysis; 5—Full paralysis; 6—Moribund/Death. Oral treatment with CF402 started at day 7 after disease induction. The clinical score was monitored daily starting with the appearance of neurological signs.

Results:

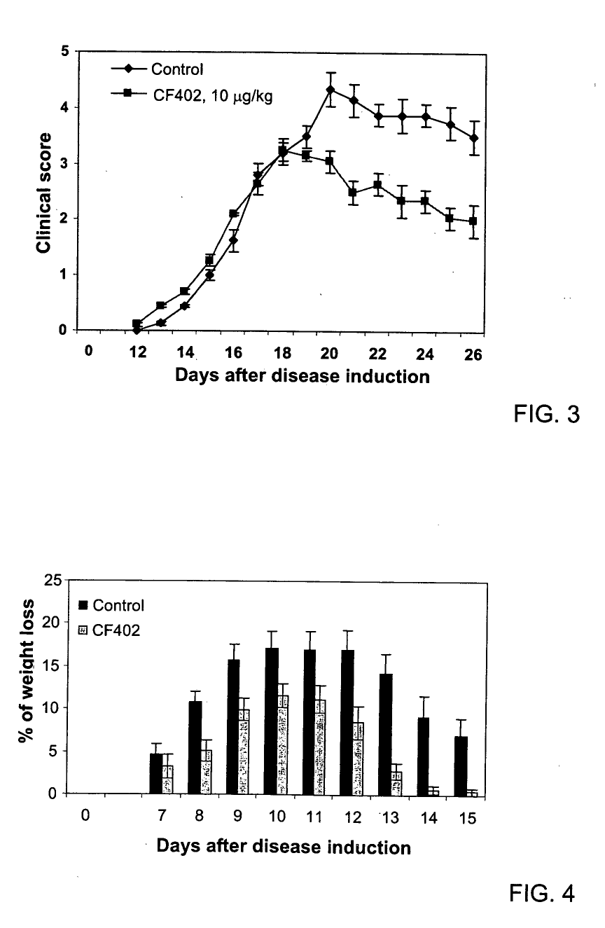

Immunization of C57BL/6J female mice with MOG resulted in clinical signs of EAE. CF402 treatment inhibited the development of the clinical signs by 40% in comparison to the control group (FIG. 3).

### EXAMPLE IV

### Effect of CF402 in a murine model of colitis

Colitis induced by dextran sodium sulfate is a murine model of intestinal inflammation that resembles human inflammatory bowel diseases such as Crohn's disease. Male Balb/C mice, 8 weeks of age were fed for 7 days, with 5% dextran sulfate sodium in distilled water throughout the experiments. CF402 was introduced at a dosage of 10 μg/kg, PO, BID starting day 4 after disease induction. Weight loss and survival were monitored.

Results

Treatment of Balb/c mice with 5% Dextran Sulfate Sodium (DSS) in their drinking water for 7 days resulted in clinical and histological signs of colitis. DSS treated mice had a marked weight loss. The CF402 treated mice had a reduced weight loss in comparison to the control (FIG. 4). Thus, CF402 treatment protected the DSS treated mice from the clinical signs of colitis.

### LIST OF REFERENCES

1. Van der Wenden, E. M., Camielli, M., Roelen, H. C. P. F., Lorenzen, A., von Frijtag Drabbe Künzel, J. K., IJzerman, A. P, J. Med. Chem., 1998, 41, 102-108.
2. Roelen, H., Veldman, N., Spek, A. L., von Frijtag Drabbe K□nzel, J., Mathot, R. A., IJzerman, A. P., J Med. Chem., 1996, 39, 1463-1471.
3. Gallo-Rodriquez, C., Ji, X., Melman, N., Siegman, B. D., Sanders, L. H., Orlina, J., Fischer, B., Pu, Q., Olah, M. E., van Galen, P. J. M., Stiles, G. L., Jacobson, K. A., J Med. Chem., 1994, 37, 636-646.
4. Van Galen, P. J. M., Van Bergen, A. H., Gallo-Rodriquez, C., Melman, N., Olah, M. E., IJzerman, A. P., Stiles, G. L., Jacobson, K. A., Mol. Pharmacol., 1994, 45, 1101-1111.
5. Van Tilburg, E. W., Von Frijtag Drabbe Künzel, J., Groote, M., Vollinga, R. C., Lorenzen, A., IJzerman, A. P., J Med. Chem., 1999, 42, 1393-1400.
6. Hutchison, A. J., Williams, M., dejesus, R., Yokoyama, R., Oei, H. H., Ghai, G. R., Webb, R. L., Zoganas, H. C., Stone, G. A., Jarvis, M. F., J. Med. Chem., 1990, 33, 1919-1924.
7. Niiya, K., Olsson, R. A., Thompson, R. D., Silvia, S. K., Ueeda, M., J. Med. Chem., 1992, 35, 4557-4561.
8. Cristalli, G., Eleuteri, A., Vittori, S., Volpini, R., Lohse, M. J., Klotz, K.-N., J. Med. Chem., 1992, 35, 2363-2368.
9. Klotz, K.-N., Camaioni, E., Volpini, R., Kachler, S., Vittori, S., Cristalli, G., Naunyn-Schmiedeberg's Arch. Pharmacol., 1999, 360, 103-108.
10. Volpini, R., Camaioni, E., Costanzi, S., Vittori, S., Klotz, K.-N., Cristalli, G., Nucleosides and Nucleotides, 1999, 18, 2511-2520.
11. Clarke, W. P., Bond, R. A., The exclusive nature of intrinsic efficacy, TiPS, 1998, 19, 270-276.
12. Kenakin, T., TiPS, 1995, 16, 188-192.
13. Fuxe, K., Ferre, S., Zoli, M., Agnati, L. F, Brain Res. Brain Res. Rev., 1998, 26, 258-273.
14. Kafka, S. H., Corbett, R., Eur. J. Pharmacol., 1996, 295, 147-154.
15. Liang, B. T., Jacobson, K. A., Proc. Natl. Acad. Sci. USA., 1998, 95, 6995-6999.
16. Jacobson, K. A., Lubitz, D. K. J. E. v., Daly, J. W., Fredholm, B. B., TiPS, 1996, 17, 108-113.

1. An anti-inflammatory pharmaceutical composition comprising as active ingredient a compound of general formula (I):
(I)
wherein
W represents oxygen or sulfur atoms;
R₁ represents lower alkyl or lower cycloalkyl;
R₂ represents halogen, alkenyl, alkynyl or alkylidenhydrazino;
R₃ represents a lower alkyl, lower cycloalkyl, aryl, (ar)alkyl or anilide, said cycloalkyl, aryl and (ar)alkyl may be substituted with one or more of the groups selected from halogen, hydroxyl, hydroxyalkyl;
and a pharmaceutically acceptable additive.

2. The pharmaceutical composition according to claim 1 for the treatment of multiple sclerosis.

3. The pharmaceutical composition according to claim 1 for the treatment of rheumatoid arthritis.

4. The pharmaceutical composition according to claim 1 for the treatment of Crohn's disease..

5. The composition of claim 1, wherein said active ingredient is a compound of formula (I) in which W represent a sulfur atom, R₁ represents an alkyl group, R₂ represents an alkynyl group and R₃ represents a hydrogen.

6. The pharmaceutical composition according to claim 5 for the treatment of multiple sclerosis.

7. The pharmaceutical composition according to claim 5 for the treatment of rheumatoid arthritis.

8. The pharmaceutical composition according to claim 5 for the treatment of colitis.

9. The composition of claim 5, wherein said active ingredient is a compound of formula (I) in which W represents a sulfur atom, R₁ represents a lower alkyl selected from the group consisting of methyl, ethyl, n- and i-propyl, R₂ represents 1-hexynyl and R₃ represents a hydrogen.

10. The pharmaceutical composition according to claim 9 for the treatment of multiple sclerosis.

11. The pharmaceutical composition according to claim 9 for the treatment of rheumatoid arthritis.

12. The pharmaceutical composition according to claim 9 for the treatment of colitis.

13. The composition of claim 9, wherein said active ingredient is 5′-deoxy-2-(1-hexynyl)-5′-methylthioadenosine.

14. The pharmaceutical composition according to claim 13 for the treatment of multiple sclerosis.

15. The pharmaceutical composition according to claim 13 for the treatment of rheumatoid arthritis.

16. The pharmaceutical composition according to claim 13 for the treatment of colitis.

17. The composition according to claim 1 for oral administration.

18. (canceled)

19. (canceled)

20. (canceled)

21. A method for treating an inflammatory disease in a subject suffering therefrom comprising administrating to said subject a pharmaceutical composition comprising as active ingredient a compound of general formula (I).

22. The method according to claim 21 wherein said composition is administered orally.

23. The method according to claim 21 for treating multiple sclerosis.

24. The method according to claim 21 for treating rheumatoid arthritis.

25. The method according to claim 21 for treating Crohn's disease.

26. The method according to claim 21 wherein said active ingredient is a compound of formula (I) in which W represent a sulfur atom, R₁ represents an alkyl group, R₂ represents an alkynyl group and R₃ represents a hydrogen.

27. The method according to claim 21 wherein said active ingredient is a compound of formula (I) in which W represents a sulfur atom, R₁ represents a lower alkyl selected from the group consisting of methyl, ethyl, n- and i-propyl, R₂ represents 1-hexynyl and R₃ represents a hydrogen.

28. The method according to claim 21 wherein said active ingredient is 5′-deoxy-2-(1-hexynyl)-5′-methylthioadenosine.
